(19) United States
(12) Patent Application Publication
(10) Pub. No.: US 2009/0317373 A1
(43) Pub. Date: Dec. 24, 2009

(54) Inhibition of the Anti-FVIII Immune Response

(75) Inventors: Srini V. Kaveri, Malakoff (FR); Sebastien Lacroix-Desmazes, Issy-Les0-Moulineaux (FR); Jagadeesh Bayry, Issy-Les-Moulineaux (FR); Suryaasrathi Dasgupta, Paris (FR); Abdessatar Chtourou, Elancourt (FR)

Correspondence Address: COHEN, PONTANI, LIEBERMAN & PAVANE LLP, 551 FIFTH AVENUE, SUITE 1210, NEW YORK, NY, 10176, US

(73) Assignees: Institut National De La Sante Et De La Recherche Medicale, Paris Cedex 13 (FR); LFB Biotechnologies, Les Ulis (FR)

(21) Appl. No.: 12/086,851

(22) PCT Filed: Dec. 22, 2006
(86) PCT No.: PCT/FR2006/002892

§ 371 Date: Feb. 17, 2009

(30) Foreign Application Priority Data
Dec. 22, 2005 (FR) 0513120

(51) Int. Cl. A61K 38/48; C12N 9/48

(52) U.S. Cl. 424/94.63; 435/212

(57) ABSTRACT

The invention relates to a compound capable of inhibiting the endocytosis of FVIII (factor VIII) by immune system cells capable of endocytosing the antigen and to the therapeutic use of such a compound for the manufacture of a medicament for use in the treatment of hemophiliacs in order to reduce the immunogenicity of FVIII and/or increase the half-life of FVIII.

The invention relates to the inhibition of the anti-FVIII immune response by blocking of the endocytosis of FVIII (factor VIII) by cells of the immune system which are capable of endocytosing the antigen.

### FIELD OF THE INVENTION

Haemophilia A is a hereditary condition associated with an anomaly in the X chromosome which manifests itself in an inability to form clots in persons affected. This disease is the result of mutations on the gene of a protein which intervenes in clotting, factor VIII (FVIII), which cause either a total absence of FVIII in the blood or a partial deficiency.

Haemophilia A is the most common deficiency affecting clotting of blood: in France it affects 1 male in 5,000 and represents 80% of patients suffering from haemophilia. The other type of haemophilia, haemophilia B, affects 20% of patients suffering from haemophilia; it is caused by a deficiency in another clotting factor, factor IX.

Current treatment of haemophilia (type A or B) consists of intravenous administration of the deficient or absent clotting factor. In France, FVIII intended for the treatment of hemophiliacs is available in the form of medicaments derived from blood supplied by Laboratoire Frangais du Fractionnement et des Biotechnologies (LFB) or from international pharmaceutical laboratories, and also in the form of recombinant medicaments produced by genetic engineering. In fact, the DNA which codes for FVIII has been isolated and expressed in mammalian cells (Wood et al., Nature (1984) 312: 330-337), and its amino acid sequence has been deduced from the cDNA.

The FVIII secreted is a glycoprotein with a molecular mass of 300 kda (2,332 amino acids) which plays a key role in the activation of the intrinsic clotting pathway. Inactive FVIII is made up of six domains: Al (residues 1-372), A2 (residues 373-740), B (residues 741-1648), A3 (residues 1649-2019), C1 (residues 2020-2172) and C2 (residues 2173-2332), from the N-terminal end to the C-terminal end. After secretion, FVIII interacts with von Willebrand factor (WF), which protects it from plasma proteases. It is in this form that FVIII circulates in the blood. FVIII dissociates from WF after cleavage by thrombin. This cleavage resulting in the elimination of the B domain and activation of FVIII in the form of a heterodimer made up of domain A1, domain A2 and the light chain A3-C1-C2. It is in this form that FVIII circulates in the plasma. This heterodimer is made up of a heavy chain (A1, A2) and a light chain (A3, C1, C2).

When it is perfused into a hemophilic patient, FVIII attaches itself to the VWF in the bloodstream of the patient. Activated FVIII acts like a cofactor of activated factor IX, accelerating conversion of factor X to activated factor X. Activated factor X converts prothrombin to thrombin. Thrombin then converts fibrinogen to fibrin and a clot forms.

The main problem encountered during administration of FVIII is the appearance of antibodies in the patient directed against FVIII, called “inhibitory antibodies”.

These antibodies neutralize the proclotting activity of FVIII, which is rendered inactive as soon as it is perfused. The clotting factor administered is thus destroyed before having been able to stop the hemorrhaging which is a serious complication of haemophilia, the treatment becoming ineffective. Furthermore, some genetically non-hemophilic patients may develop inhibitors against endogenous FVIII: this is an acquired haemophilia.

The mechanisms by which the anti-FVIII antibodies interfere with the function of FVIII are numerous, and include interference in the proteolytic cleavage of FVIII and in the interaction of FVIII with various partners, such as von Willebrand factor (VWF), phospholipids (PL), factor IX, activated factor X (FXa) or APC (activated protein C).

The first stage in the initiation of a specific immune response to FVIII is endocytosis of FVIII by antigen-presenting cells. Dendritic cells (DC) are the most potent antigen-presenting cells (APC), and one of the rare types of APC capable of activating primary naïve T cells. The DCs are thus capable of initiating a specific immune response to the antigen (1,2). The DCs endocytose the antigen by the intermediary of a receptor or by macropinocytosis; endocytosis mediated by a receptor being advantageous for the DCs in vivo.

The surface of the DC has numerous endocytic receptors, the majority of which are dependent on divalent ions, chiefly calcium (FIG. 1). Numerous endocytic receptors, because of their carbohydrate recognition domain (CRD), are specific for sugar residues present on the antigens, and are called C-type lectin receptors (CLR). Mannose residues on an antigen can thus be recognized by a series of mannose-sensitive CLRs on the surface of the dendritic cell which contains the mannose receptor (MR, CD206), the DC-SIGN receptor (CD209), dectin, DEC-205 (CD205). Mannan is a ligand for these mannose-sensitive CLRs which are sensitive to mannose, in particular for MR and DC-SIGN (3-5). The DC-SIGN molecule of dendritic cells attaches itself to ICAM-3 molecules of T lymphocytes. This specific interaction seems to play an important role in the initiation of the immunological synapse between dendritic cells and TLs. The activation of lymphocytes is inhibited by an anti-DC-SIGN blocking antibody.

The FVIII molecule contains 25 consensus sequences (Asn-Xxx-Thr/Ser) which are potential glycosylation sites bonded to N, 20 of which have been demonstrated to be glycosylated (6). Some glycosylations bonded to N are maintained on FVIII-BDD (B domain-deleted recombinant FVIII, Refacto, Wyeth) (7). Both FVIII derived from plasma and recombinant FVIII have shown similar glycosylation profiles, with residues ending in a galactose or a mannose (8, 9).

FVIII and FVIII-BDD are thus candidate ligands for CLRs on the surface of the dendritic cell.

### PRIOR ART

There are several treatments which enable the consequences of the anti-FVIII immune response to be attenuated, such as, for example, treatments involving desmopressin, which is a synthetic hormone which stimulates FVIII production, agents which promote clotting, such as prothrombin complex concentrates or activated prothrombin complex concentrates, recombinant factor VIIa and perfusions of significant or intermediate amounts of FVIII to induce tolerance. However, these methods remain very costly and are not very effective.

Another more recent strategy of combating inhibitory antibodies of FVIII envisages the administration of anti-idiotypical antibodies (antibodies having the ability to interact with the variable region of other antibodies), neutralizing the inhibitory antibodies (Saint-Remy J M et al. (1999) Vox Sang; 77 (suppl 1): 21-24). Because of the complexity of in vivo analysis of this polyclonal immune response, teams have isolated monoclonal antibodies directed against certain domains of FVIII. A human monoclonal antibody of the IgG4kappa type, LE2E9, has thus been isolated. This antibody is directed against domain C1 of FVIII and inhibits the cofactor activity of FVIII and its binding to vWF (Jacquemin et al. (2000) Blood 95:156-163). In the same way, a human monoclonal antibody directed against domain C2 of FVIII, called BO2C11 (IgG4kappa), produced from the memory B cell repertoire of a patient suffering from haemophilia A with inhibitors, has been isolated (Jacquemin et al. Blood 1998 Jul. 15; 92 (2):496-506). BO2C11 recognizes domain C2 of FVIII and inhibits its binding to VWF and to phospholipids. It completely inhibits the proclotting activity of native and activated FVIII. Another example of a monoclonal antibody is the antibody BOIIB2 directed against domain A2 of FVIII. The antibody BOIIB2 inhibits to 99% the activity of FVIII. By binding to domain A2, it can interfere in and inhibit the attachment of FIXa, which has an attachment site of low affinity in this region of FVIII, and from then inhibits the enzymatic activity of FIXa. The second mode of action which can be envisaged is its interference in the equilibrium between the heterodimer form (A2:A1 and A3:C1:C2) of FVIII and the heterotrimer form (A2 and A1 and A3:C1:C2) of FVIII by accelerating dissociation of domain A2 from these complexes, rendering them non-functional (Ananyeva N M et al. (2004) Blood Coagul Fibrinolysis, March; 15(2):109-24. Revue).

However, the anti-FVIII immune response is polyclonal, and the FVIII inhibitory antibodies developed by a patient are not necessarily all directed against a single domain of FVIII.

A treatment consisting of the administration of anti-idiotypical antibodies directed against anti-FVIII antibodies directed against a single domain of FVIII could only partly neutralize the anti-FVIII immune response developed in the patient.

No treatment is available for action before the appearance of the anti-FVIII immune response and therefore for avoiding it.

### SUMMARY OF THE INVENTION

To mitigate such disadvantages of the prior art, the Applicant has found, surprisingly, that it is possible to block the endocytosis of FVIII by cells of the immune system which are capable of endocytosing the antigen and consequently of inhibiting the formation of anti-FVIII inhibitory antibodies and to increase the half-life of FVIII.

According to one aspect of the present invention, a deglycosylated factor VIII or a fragment thereof is provided, for which the ability for interacting and the ability for being endocytosited by cells which are capable of endocytosing an antigen are decreased or inhibited with respect to native factor VIII.

According to another particularly advantageous embodiment, this deglycosylated factor VIII or the fragment thereof has a reduced or inhibited ability for interaction with the receptors present on the surface of the said cells which are capable of endocytosing an antigen, in particular with receptors specific for mannose, and more particularly with the mannose receptor CD206 or the DC-SIGN receptor CD209 (dendritic cell-specific intercellular adhesion molecule 3 (ICAM-3)-grabbing non-integrin).

According to a preferred form of the present invention, the said cells which are capable of endocytosing an antigen are antigen-presenting cells (APCs) and, in particular, dendritic cells or B lymphocytes. According to another preferred form of the invention, the said cells which are capable of endocytosing an antigen are chosen from macrophages, endothelial cells, liver sinusoidal endothelial cells, liver Kupffer cells.

According to a preferred embodiment of the present invention, the factor VIII or the fragment thereof is deglycosylated without one or more of the enzymes chosen from the groups consisting of a neuraminidase, a beta-galactosidase and an alpha-mannosidase having been used.

According to a particular embodiment of the invention, the factor VIII or the fragment thereof is deglycosylated by the action of a single enzyme of the endoglucosidase type, and in particular of the endo-beta-N-acetylglucosaminidase type. In a preferred form of the present invention, the enzyme used has the capacity for cutting carbohydrate structures of the oligomannose type and of the hybrid type, but does not have the capacity for cutting carbohydrate structures of the complex type.

In a particular embodiment, the said enzyme of the endo-beta-N-acetylglucosaminidase type is chosen from the group consisting of endo-beta-N-acetylglucosaminidase F1 and endo-beta-N-acetylglucosaminidase H, for example the endo-beta-N-acetylglucosaminidase F1 of Chryseobacterium (Flavobacterium) meningosepticum or the endo-beta-N-acetylglucosaminidase H of Streptomyces picatus.

According to another embodiment of the present invention, the deglycosylated factor VIII of the invention or the fragment thereof is used as a medicament, in particular for the treatment of hemophilic patients, and especially for the treatment of haemophilia of type A or of type B. According to another embodiment of the present invention, the deglycosylated factor VIII of the invention or the fragment thereof is also used for the preparation of a medicament intended for the treatment of hemophilic patients or for the treatment of haemophilia of type A or type B, and can advantageously be used in combination with exogenous factor VIII.

According to yet another object of the present invention, the deglycosylated factor VIII of the invention or the fragment thereof can be used for the preparation of a medicament intended for increasing the half-life of an exogenous factor VIII or intended for reducing the immunogenicity of an exogenous factor VIII in hemophilic patients.

Another aspect of the present invention also relates to a pharmaceutical composition comprising at least the deglycosylated factor VIII of the invention or a fragment thereof, as well as one or more pharmaceutically acceptable adjuvant(s) and/or excipient(s).

A last object of the invention relates to the use of a composition comprising mannan for the preparation of a medicament intended for the treatment of hemophilic patients, or for the treatment of haemophilia of type A or B. According to a particular embodiment, this composition comprising mannan is used for the preparation of a medicament intended for increasing the half-life of an exogenous factor VIII or for reducing the immunogenicity of an exogenous factor VIII in hemophilic patients. According to an advantageous embodiment, the mannan composition also comprises an exogenous factor VIII of the native type and/or a deglycosylated factor VIII or a fragment thereof according to the invention.

Generally, both in the description and abstract and in the claims, the following terms encountered have the following meanings, unless stipulated otherwise:

Generally, both in the description and abstract and in the claims, the definition of a carbohydrate chain bonded to an asparagine residue (N) (which residue can be contained in a polypeptide, such as, for example, native or deglycosylated FVIII) is well-known to the person skilled in the art and has a base structure consisting of two N-acetyl-glucosamines (GlcNAc) and three mannoses, and other monosaccharides may become grafted onto this structure. The base structure is formed by linking together two N-acetylglucosamine (GlcNAc) residues bonded in position β1,4. The one forms the N-glycosidic bond with the protein (by asparagine). The other is bonded to a mannose residue in position β1,4, itself bonded to 2 mannose residues, in position α1,6 and α1,3; for example:

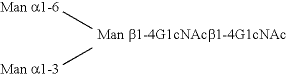

The carbohydrate chains bonded to an asparagine shown in the present Application are given purely by way of example and must not be regarded as limiting the present invention in any manner whatsoever.

“Antennae” are formed by the addition of monosaccharides onto the terminal mannoses. Depending on the nature of the sugar, a distinction is thus made between three types of glycan structures:
structures of the “oligomannose” type, which correspond to the addition of mannose residues onto the base structure; for example:

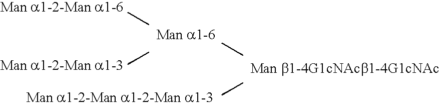

structures of the “complex” type, which result from bonding of N-acetyllactosamine residues (LacNac: Gal β1,4 GlcNAc) onto the terminal mannoses of the base structure (the addition of GlcNAc and Gal is sequential and results from activities of GlcNAcT-1,2,3,4 and 5 and then of GalT); for example:

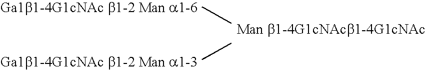

structures of the “hybrid” type, which are, as their name indicates, a mixture of the two preceding forms. In the example given below, the mannose of the α1,6 branch is substituted solely by mannose residues, while the α1,3 branch is substituted by one or two N-acetyllactosamine residues:

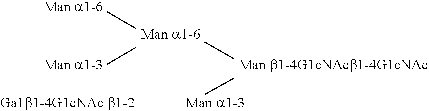

### LEGEND TO THE FIGURES

FIG. 1: Diagrammatic representation of FVIII and receptors of the DC membrane FIG. 2a: Measurement of the difference between the conditions at 37° C. and 42° C. of internalization of FVIII-BDD (-◯-) and whole FVIII (--)

FIG. 2b: Measurement of the internalization of FVIII and FVIII-BDD as a function of time at 4° C. or 37° C.

FIG. 2c: Measurement of the relative internalization of FVIII (%) in the presence of medium, EDTA, mannan or galactose

FIG. 2d: Measurement of the relative internalization (%) of FVIII, FVIII-BDD, dextran and ly in the presence or absence of mannan

FIG. 2e: Measurement of the inhibition of the internalization (%) of FVIII, FVIII-BDD and dextran as a function of an increasing concentration of mannan

FIG. 2f: Measurement of the internalization of α2M in the presence or absence of RAP or mannan

FIG. 3a: Measurement of the relative internalization (%) of FVIII and FVIII-BDD in the presence of anti-MR and anti-DC-SIGN antibody

FIG. 3b: Measurement of the internalization (%) of FVIII by HD420 cells as a function of the concentration of FVIII (μM)

FIG. 3c: Measurement of the intensity of binding to the constructs CTLD1-3 and CTLD4-7 as a function of the concentration of FVIII, FVIII-BDD and mannan (μg/ml)

FIG. 3d: Measurement of the inhibition in % of the binding of FVIII and FVIII-BDD to the constructs CTLD1-3 and CTLD4-7 as a function of the concentration (μg/ml) of mannan

FIG. 4a: Measurement of the proliferation of CD4+ T cells (cpm) as a function of the CD4+ T:DC ratio

FIG. 4b: Measurement of the activation of the human anti-FVIII TCD4+ clone D9E9 by measurement of the production of IFN-gamma

FIG. 5a: Measurement of the inhibition of the proclotting activity of FVIII (%)

FIG. 5b: Measurement of the inhibition of the binding of FVIII to VWF (%) by mannan

FIG. 6: Western blot of the detection of sugars on FVIII

FIG. 7: Western blot of the detection of FVIII by CTLD4-7-Fc

FIG. 8: Measurement of the inhibition of the internalization of FVIII (%) after treatment with Endo-F1 or without treatment

FIG. 9: Measurement of the binding of CTLD(4-7)-Fc to FVIII-BDD (OD 492 nm) as a function of the concentration of VWF.

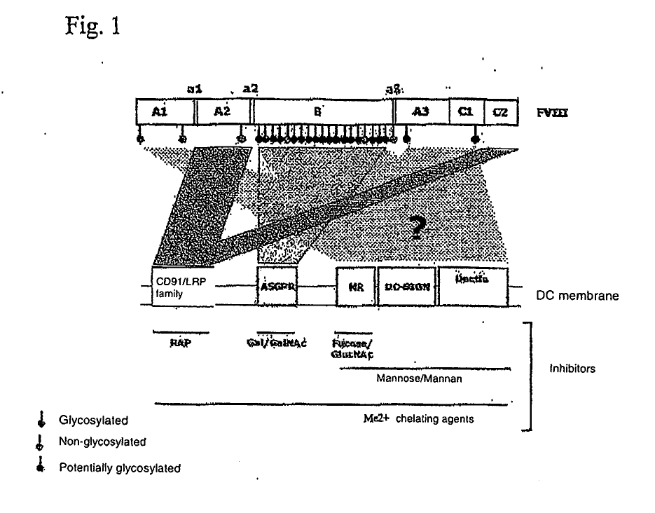

### DETAILED DESCRIPTION OF THE INVENTION

The present invention chiefly relates to a compound which is capable of inhibiting the interaction of factor VIII with cells which are capable of endocytosing the antigen and the endocytosis of factor VIII by the said cells.

In fact, the Applicant has demonstrated that certain compounds are capable of inhibiting the endocytosis of FVIII by dendritic cells, such as mannan and demannosylated FVIII.

The Applicant has also demonstrated that mannan thus inhibits the proliferation of T cells specific for FVIII without blocking the proclotting activity of FVIII or the interaction of FVIII with VWF.

In particular, this compound may be capable of inhibiting the interaction of factor VIII with a receptor which is present on the cells which are capable of endocytosing the antigen and is responsible for the endocytosis of factor VIII by the said cells.

The present invention thus also includes both compounds other than FVIII which block the receptors on the cells responsible for the endocytosis of FVIII, and a modified FVIII molecule, in its entirety or a fragment thereof, the ability of which for binding with the cells responsible for its endocytosis is lower than that of native FVIII. Each of these two types of compound thus inhibits the endocytosis of FVIII by cells of the immune system which are capable of endocytosing the antigen.

In the case of a modified FVIII molecule or a fragment thereof, the said FVIII compound inhibits its own interaction and its own endocytosis by the cells which are capable of endocytosing thereof: it is therefore a modified FVIII or a fragment thereof, the endocytosis of which is reduced with respect to native FVIII. Preferably, this FVIII is modified with respect to native FVIII at the level of its glycosylation.

In a preferred form of the invention, the said receptor is specific for mannose, and particularly preferably the said receptor which is specific for mannose is the mannose receptor CD206 or the DC-SIGN (dendritic cell-specific intercellular adhesion molecule 3 (ICAM-3)-grabbing non-integrin) receptor CD209.

In a particular embodiment of the invention, the said cells which are capable of endocytosing the antigen are antigen-presenting cells, for example dendritic cells or B lymphocytes.

The compound according to the invention is thus capable of inhibiting the endocytosis of FVIII by antigen-presenting cells, and therefore of inhibiting the presentation of FVIII peptides which initiates the immune response. Consequently, the compound according to the invention thus inhibits the production of the anti-FVIII antibody and reduces the immunogenicity of FVIII.

In a particular embodiment of the invention, the said cells which are capable of endocytosing the antigen are chosen from macrophages, endothelial cells, liver sinusoidal endothelial cells, liver Kupffer cells.

These cells endocytose FVIII with the aim of eliminating it. By inhibiting the endocytosis of FVIII by these cells, the compound according to the invention reduces this elimination route of FVIII, increases the amount of circulating FVIII and therefore increases the half-life of FVIII.

The invention also relates to the use of a compound according to the invention for the preparation of a medicament intended for the treatment of hemophiliacs in combination with exogenous FVIII.

In particular, this medicament is intended for reducing the immunogenicity of exogenous FVIII in hemophilic patients and/or increasing the half-life of exogenous FVIII in hemophilic patients.

In this case, the medicament is administered with exogenous FVIII or a fragment thereof for its proclotting activity.

In particular, mannan is used for the preparation of a medicament intended for the treatment of hemophiliacs in combination with exogenous FVIII, by reducing the immunogenicity of exogenous FVIII in hemophilic patients and/or by increasing the half-life of exogenous FVIII in hemophilic patients.

The invention additionally relates to the use of a modified FVIII or of a fragment thereof, the endocytosis of which by cells which are capable of endocytosing the antigen is reduced with respect to native FVIII, for the preparation of a medicament intended for the treatment of hemophiliacs.

In particular, this FVIII or fragment of FVIII is demannosylated.

In this case, the said medicament comprises an FVIII which is less immunogenic than native FVIII, the half-life of which is increased with respect to native FVIII, but which has retained its proclotting activity in full.

The invention additionally relates to a pharmaceutical composition comprising at least one compound according to the invention and one or more pharmaceutically acceptable adjuvant(s) and/or excipient(s).

The following examples illustrate the invention without limiting its scope.

### Example 1

### Preparation of Human DCs Derived from Monocytes

DCs were prepared from monocytes as described previously (29) with a change in the culture media. Briefly, mononucleated cells were isolated from heparinized leukoplatelet layers (“buffy coats”) from healthy adult donors by adhesion on plastic cell culture dishes in RPMI 1640 medium supplemented with 10% human AB serum, glutamine and antibiotics for 60 minutes. Non-adhering cells were removed by 3 gentle washings with the medium. The adhering monocytes were cultured in X-VIVO 15 medium (Cambrex Bio Sciences, Paris, France) supplemented with 1% human AB serum and antibiotics and in the presence of 500 IU/ml of recombinant human interleukin-4 (rhIL-4), R&D Systems (Lille, France) and 1,000 IU/ml of recombinant human granulocyte macrophage colony-stimulating factor (rhGM-CSF), Immunotools (Friesoythe, Germany). Half of the medium, comprising all the supplements, was replaced every two days. After culture for 5 days, the non-adhering cells and those with little adhesion, corresponding to the fraction enriched in DC, were harvested, washed and used for subsequent experiments.

### Example 2

### Conjugation of Whole Recombinant Human FVIII and B Domain-Deleted Recombinant Human FVIII with Fluorescein

Whole recombinant human FVIII (1,000 IU, Kogenate, Bayer) or B domain-deleted recombinant human FVIII (FVIII-BDD) (1,000 IU, Refacto, Wyeth) were solubilized in water and dialyzed against bicarbonate buffer (pH 9.2) containing CaCl₂, 5 mM at 4° C., followed by coupling to fluorescein 5-isothiocyanate (isomer I, Sigma-Aldrich, Saint Quentin Fallavier, France) for 7-8 hours at 4° C. The labelled FVIII was then dialyzed against RPMI 1640 medium to remove non-coupled FITC. The FVIII-FITC was quantified by the Bradford method using bovine serum albumin as the standard.

### Example 3

### Binding of Constructs of the Mannose Receptor to FVIII using ELISA

Constructs of the mannose receptor, CTLD(4-7)-FC and CR-FNII-CTLD(1-3)-CR-Fc, were kindly donated by Dr Luisa Martinez-Pomares, School of Molecular Medical Sciences, Queen's Medical Centre, University of Nottingham, UK. The binding of the constructs to whole recombinant human FVIII and to B domain-deleted recombinant human FVIII was tested either directly by measuring the binding to ELISA plates coated with ligands (mannan was used as a positive control) or indirectly by inhibition tests. The ELISA plates (Nunc; MAXISORP) were coated overnight with dilution series of the FVIII forms (starting at 50 μg/ml) in 154 mM NaCl. TTBS buffer (Tris-HCl 10 mM, pH 7.5, Ca²⁺ 10 mM, NaCl 154 mM and 0.05% Tween-20) was used for all the washings. The non-reactive sites were blocked by TBS buffer (TTBS without Tween-20) containing 3% BSA. The plates were then incubated with 2 or 10 μg/ml of the constructs for 2 h at room temperature in TTBS buffer containing 3% BSA. The binding of the MR constructs was detected using a mouse antibody specific for the Fc part of human IgG conjugated to HRP (clone JDC-10, Southern Biotechnology Associates, Inc. AL, USA). The activity of the HRP was demonstrated with the substrate OPD (o-phenylenediamine, Sigma). The absorbance was measured at 492 nm. For the inhibition tests, the plates were coated with each form of FVIII (5.56 μg/ml). Various concentrations of mannan were incubated with 10 μg/ml of the construct CTLD(4-7)-Fc for 30 min at room temperature before incubation with the plates coated with FVIII. The absorbance was measured as described above.

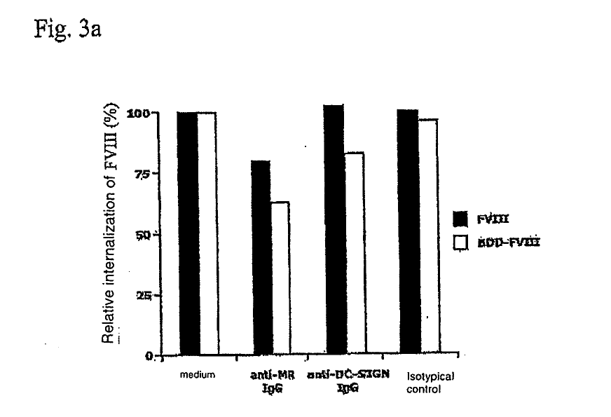

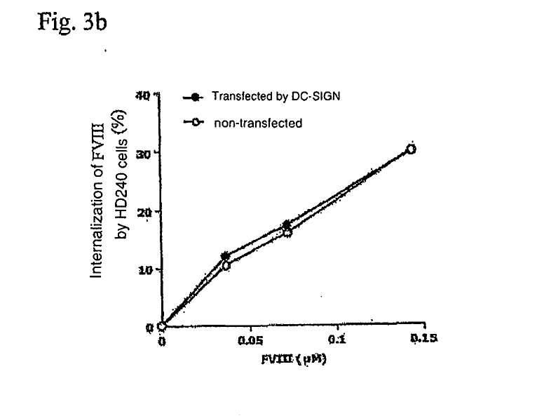

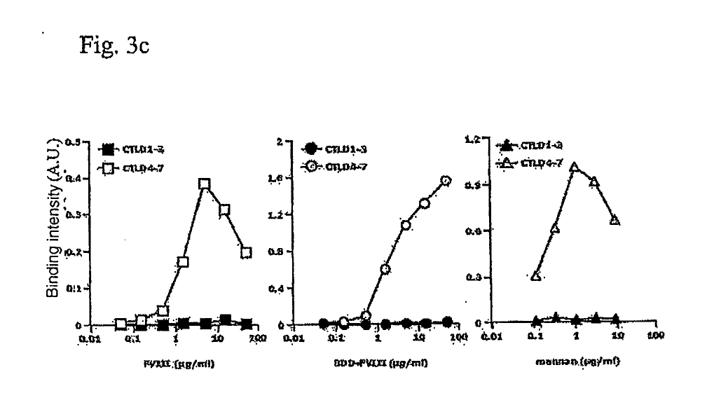

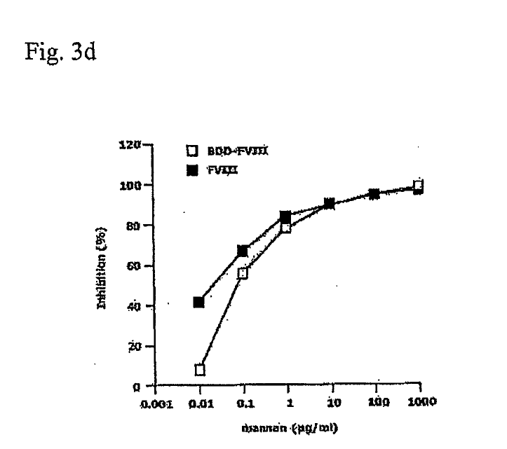

### Results: FIGS. 3c and 3d

The mannose receptor contains 8 C-type lectin domains (CTLDs): CTLDs 4 to 7 have an affinity for mannosylated architectures, but not CTLDs 1 to 3.

As shown in FIG. 3c, mannan shows specific binding to CTLD(4-7)-Fc. Both FVIII and FVIII-BDD show a dose-dependent interaction specifically with CTLD(4-7)-Fc, whereas they do not bind to the construct CTLD(1-3), indicating that glycosylation (probably the mannosylation shown) outside domain B of FVIII allows the molecule to interact with the MR.

The mannan preincubated with CTLD(4-7)-Fc inhibits the binding of the two forms of FVIII to CTLD(4-7)-Fc in a dose-dependent manner. This could reflect the situation of DCs where the MR on the DCs are saturated with mannan, thus inhibiting the endocytosis of FVIII.

### Example 4

### In Vitro Test of the Internalization of FVIII by Human DCS Derived from Monocytes

### Material and Methods

The DCs obtained in Example 1 (0.4×10⁵ cells/well of a 96-well plate with 100 μl/well) were incubated with various doses (0.029, 0.057, 0.143 and 0.358 μM) of fluorescent conjugated ligands (FVIII-FITC, BDD-FVIII-FITC) obtained in Example 2 in X-VIVO medium for 0, 15, 60 and 120 min at 4° C. or at 37° C. After the incubation period, the cells were washed with cold PBS and analyzed by flow cytometry. To investigate the involvement of receptors in the internalization, the cells were preincubated for 30 min at 37° C. with 0, 0.001, 0.01, 0.01 and 1 mg/ml of mannan (Sigma-Aldrich, Saint Quentin Fallavier, France) before the addition of ligands conjugated to fluorescein. In addition to FVIII-FITC and FVIII-BDD-FITC, human α2-macroglobulin-MA (α2M) conjugated to FITC from Biomac (Leipzig, Germany), dextran-FITC (molecular weight 40,000) from Molecular Probes (Leiden, The Netherlands) and Lucifer yellow (LY-CH) from Sigma-Aldrich were used. The investigation of the internalization of FVIII was also carried out in the presence of 20 μg/ml of anti-mannose receptor monoclonal antibody (PAM-1, isotype IgG1) or anti-DC-SIGN monoclonal antibody ((AZN-D1, isotype IgG1) or mouse IgG_{λ,k} conjugated to PE-Cy (BD Pharmingen, France). To investigate whether DC-SIGN is involved in the endocytosis of FVIII, the B lymphocyte line HD-420 transformed by wild-type EBV and transfected with DC-SIGN or not transfected was incubated with various concentrations of FVIII-FITC (0, 0.036, 0.072 and 0.143 μM) for 2 hours in RPMI 1640 medium supplemented with 10% FCS (foetal calf serum) and antibiotics. The analysis was carried out as for the DCs.

### Results

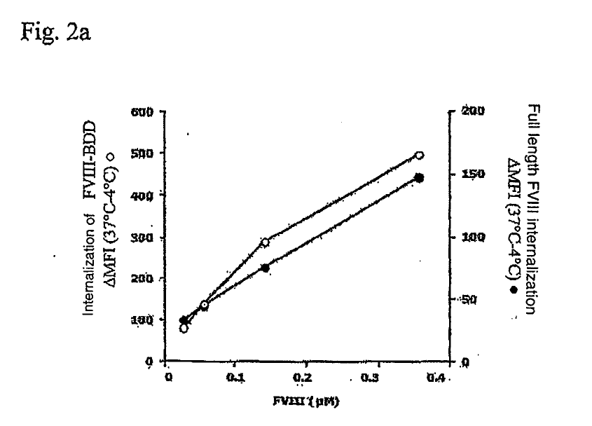

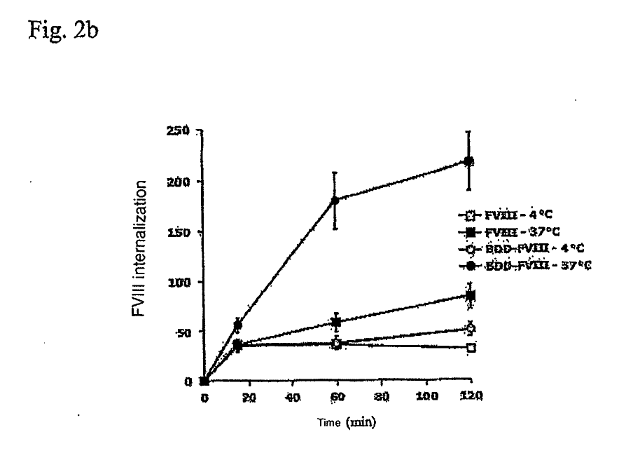

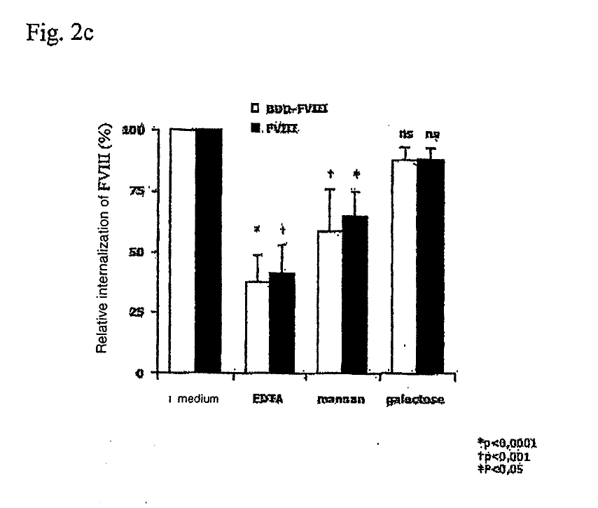

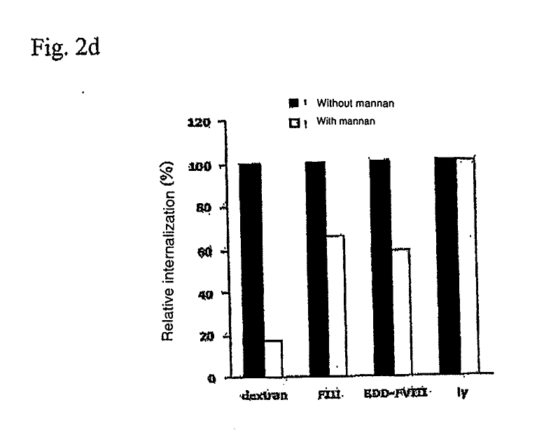

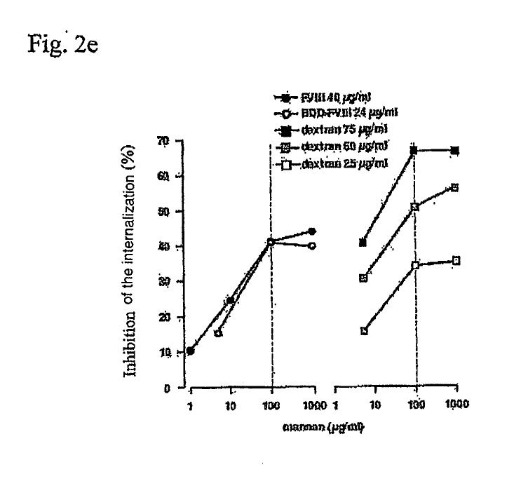

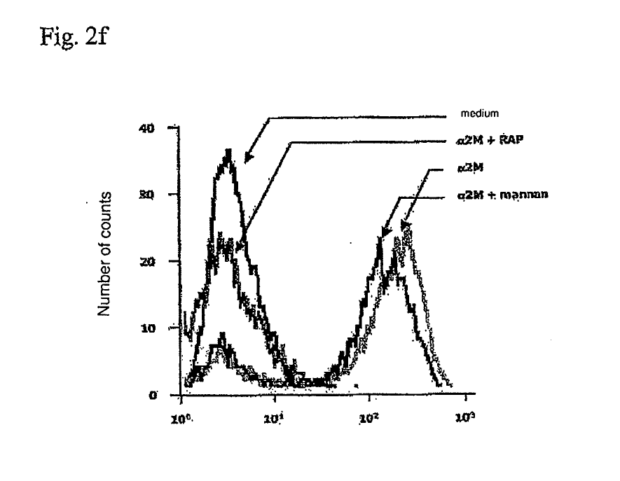

### FIG. 2a, b

The DCs internalize whole FVIII and FVIII-BDD proportionally to the dose and time. The ΔMFI values calculated represent the different labelling of DCs positive towards FVIII after incubation for 2 hours at 37° C. and 4° C. The results demonstrate that the internalization of FVIII is an active process. An incubation period of 2 hours and a concentration of ligand of 0.143 μM were chosen for the subsequent experiments.

### FIG. 2c

The internalization of FVIII by immature DCs was reduced significantly by preincubating the cells with 5 mM EDTA (58.1±11.1% and 62.4±11.4% inhibition for whole FVIII and FVIII-BDD respectively), thus demonstrating a role of receptors dependent on divalent ions. The possible FVIII receptors on the DCs (i.e. CD91/LRP, ASGPR, mannose receptors) were investigated using specific competitive ligands. The receptor-associated protein (RAP) of 38 kD blocks the endocytosis of ligands by members of the LDL receptor family, such as CD91/LRP. An excess of RAP did not allow the endocytosis of FVIII by the DCs to be prevented. Furthermore, D-galactose, a competitive ligand for the C-type lectin receptor ASGPR, did not significantly reduce the internalization of FVIII, independently of the presence or absence of domain B. In contrast, the addition of mannan (1 mg/ml) significantly reduced the endocytosis of FVIII (35.0±10.0% and 41.3±17.2% inhibition for whole FVIII and FVIII-BDD respectively; p<0.05). This indicates that mannose-sensitive CLRs are involved directly or indirectly in the internalization of FVIII.

### FIG. 2d

The specificity of mannan for mannose-sensitive CLRs was confirmed using dextran, a typical ligand of the mannose-sensitive CLRs, and Lucifer yellow (ly), internalization of which proceeds exclusively by macropinocytosis independently of any receptor. The internalization of dextran was blocked to 80% in the presence of mannan, whereas that of ly was not affected.

### FIG. 2e

The neutralizing effect of mannan on the internalization of the antigen is proportional to the dose for FVIII, FVIII-BDD and dextran, regardless of the antigen concentration. Interestingly, a similar saturation concentration of mannan was reached for the various antigens (100 μg/ml), suggesting that mannan-sensitive endocytosis of these antigens proceeds via similar mannose-sensitive receptors.

### FIG. 2f

FVIII can interact with various endocytic receptors.

Activated α2M, a mannosylated protein which specifically targets the endocytic receptor CD91/LRP, was used as a model antigen. It was found that the expression of mannosylated residues on α2M does not influence the internalization of the antigen if other endocytic receptors are involved. Thus, the internalization of α2M was inhibited completely in the presence of an excess of RAP, whereas mannan showed no effect.

These results suggest indirectly that the mannosylated parts present on FVIII are unique and render the FVIII more attractive to antigen-presenting cells (APC) than other mannosylated antigens.

### FIGS. 3a and 3b

Several receptors on the surface of DCs and other APCs are sensitive to mannan, including the mannose receptor MR (CD206) and the DC-SIGN receptor (CD209). The antibody PMA-1 (anti-MR) inhibited the internalization of FVIII by 20.22% and that of FVIII-BDD by 37.35%, thus indicating the involvement of the MR in the endocytosis of FVIII. The fact that the effect of mannan on the endocytosis of FVIII had been more pronounced than that of the anti-MR monoclonal antibody can be explained by the fact that the internalization of FVIII by DCs involves several receptors sensitive to mannan which have not yet been characterized. On the other hand, the increased inhibition with mannan may result from the fact that the MR has several carbohydrate recognition domains (CRDs): inhibition is therefore more effective by polycarbohydrated mannan than by the antibody PAM-1, which is directed only against the 4th CRD of the MR.

The antibody AZN-D1 (anti-DC-SIGN) did not inhibit the internalization of FVIII by immature DCs, whereas that of FVIII-BDD was inhibited by 17.6%. The expression of DC-SIGN by transfected B cells did not allow an increase in the endocytosis of FVIII.

These data suggest that DC-SIGN plays a minor role in the internalization of FVIII by human DCs.

### Example 5

### In Vitro Test of the Proliferation of Autologous CD4+ T Cells

The DCs of Example 1 were incubated with mannan (1 mg/ml) for 30 min at 37° C. FVIII dialyzed against RPMI 1640 medium (Kogenate, Bayer) was added (40 μg/ml, 0.143 μM) for 2 hours. The cells were then washed and incubated with LPS (1 μg/0.5 million cells) in complete medium for 48 hours. The cells were then washed and cultured (RPMI 1640 medium, supplemented with 10% male human AB serum, in 96-well cell culture plates with a round base with 200 μl per well) with autologous CD4+ T cells obtained from the PBMC of the corresponding donor using the MACS cell isolation kit (Miltenyi Biotech, Bergisch Gladbach, Germany). The number of CD4+ T cells was kept constant in each well (100,000 cells/well), while the number of DCs varied (5,000, 10,000 and 15,000 DCs in triplicate), giving a T:DC cell ratio of 20:1, 10:1, 6.6:1.

After culture for 4 days, 0.5 μCi of tritiated thymidine was added to each well. The cells were harvested after 16 hours and the radioactivity incorporated was counted.

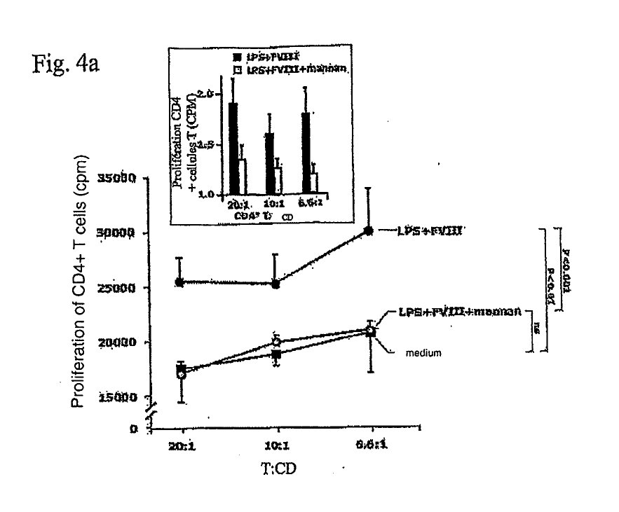

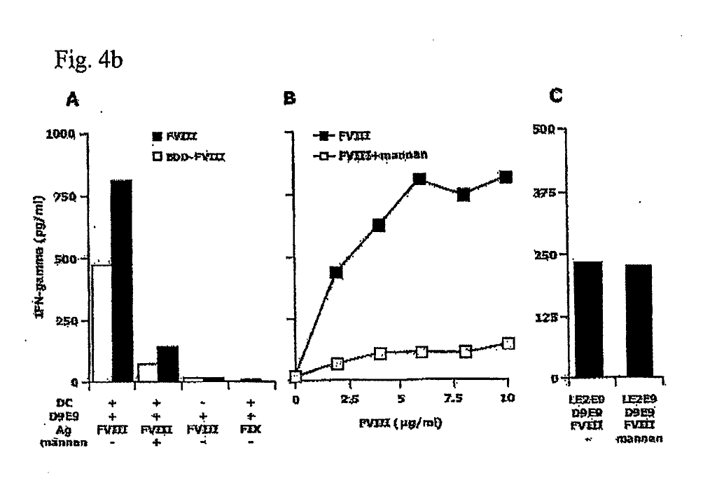

### Results: FIG. 4a

Mannan reduces the endocytosis of FVIII by the human DCs by up to 40%.

FIG. 4a: The DCs according to Example 1 are incubated by themselves or with mannan before the addition of FVIII. After maturation in the presence of LPS, the DCs are incubated with autologous CD4+ T helper cells. Mannan reduces the proliferation of T cells to levels comparable to those of negative controls.

### Example 6

### In Vitro Test of the Activation of Clones of T Cells Specific for FVIII

After washing, the DCs of Example 1 were resuspended in DMEM:F12 (1:1) medium containing 10% FCS and 10,000 DCs were introduced into each well of a 96-well cell culture plate with a round base. After incubation with mannan (1 mg/ml) in each well for 30 min, the DCs were cultured with 5,000 D9E9 (T cell clone specific for FVIII) in DMEM:F12 (1:1) medium containing 10% FCS and 20 U/ml of rhIL-2 with various doses of whole FVIII or FVIII-BDD (10, 8, 6, 4, 2 μg/ml) for 20 hours at 37° C. Like the D9E9 cells, the LE2E9 and BO2C11 lines of B cells transformed by EBV were cultured in the presence of 10 μg/ml of whole FVIII or FVIII-BDD. The control conditions were maintained in the absence of FVIII, D9E9 or DCs. As a negative control, hrFIX (Benefix, Baxter) was incubated with the cells at isomolar concentrations of FVIII. The supernatants were harvested at the end of the incubation period and tested for their production of IFNγ using the human IFN-γ Duo Set (DY285, R&D Systems) in accordance with the manufacturer's instructions.

### Results: FIG. 4b

FVIII induces a dose-dependent activation of D9E9 cells which is inhibited by up to 84% in the presence of mannan (B). The proliferation of T cells is specific, as indicated by the absence of proliferation in the presence of FIX (A).

Mannan does not prevent activation of D9E9 cells by autologous B cells pulsed with FVIII, LE2E9 (C). This suggests that the potential effect of mannan in reducing the immunogenicity of FVIII may be exploited therapeutically solely in a situation where the B cells specific for FVIII have not yet been stimulated (i.e. in patients who have not previously been treated), or the inhibitor has not yet developed after treatment.

### Conclusion:

The reduction in the internalization of FVIII induced in the presence of manna results in a decreased presentation of peptides derived from FVIII to CD4+ T cells, and consequently in a weaker activation of T lymphocytes.

### Example 7

### Clotting Test

Normal human plasma was incubated with an identical volume of mannan (0 to 4,000 mg/ml) for 2 hours at 37° C. The residual FVIII activity was measured in a one-stage clotting test using human placental prothrombin as an activator (Dade Behring Marburg GmbH, Marburg, Germany) and FVIII-depleted plasma (Dade Behring Marburg GmbH, Marburg, Germany) as a substrate and a fibrin timer (Sysmex CA500, Dade Behring). The dilutions were performed in Owren-Koller buffer (Diagnostica Stago, Asnieres, France).

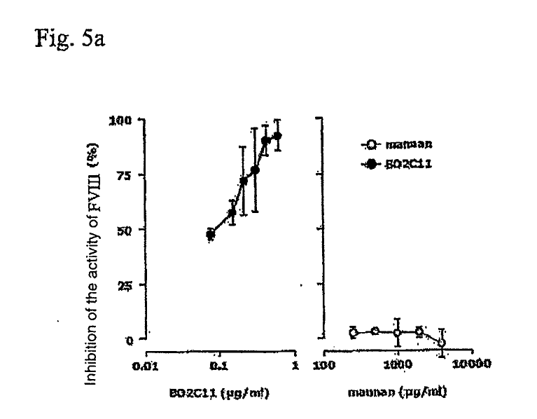

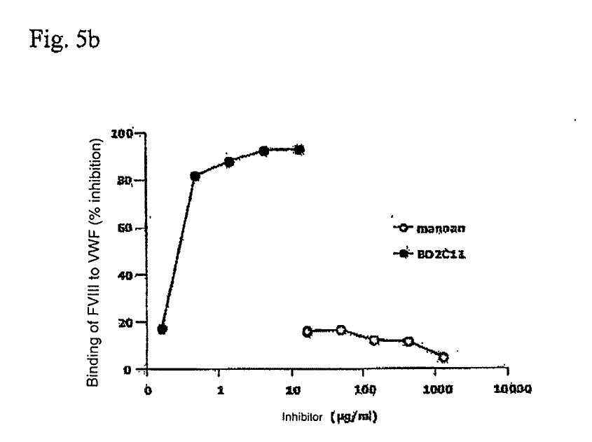

### Results: FIG. 5a

Mannan does not block the proclotting activity of FVIII.

### Example 8

### ELISA for Binding of FVIII to VWF

ELISA plates (Nunc, Roskilde, Denmark) were coated with VWF (VWF, Willefectin, LFB, Les Ulis, France) at 2 μg/ml per well in PBS (pH 7.4) at 37° C. for 1 hour. The plates were saturated with PBS containing 1% skimmed milk and 0.1% Tween 20 for 1 hour at 37° C. FVIII (0.3 μg/ml) was preincubated by itself or with mannan (0.01 to 4 mg/ml) or with BO2C11 (human monoclonal anti-FVIII IgG) (0.05 to 40 μg/ml) in a blocking buffer for 1 hour at 37° C. and then added to the wells coated with VWF and incubated for 1 hour at 37° C. A mouse monoclonal anti-FVIII IgG (mAb6) (3 μg/ml) was incubated at 37° C. for 1 hour. The reactivities were revealed with rabbit anti-mouse IgG antibodies coupled to streptavidin peroxidase (Jackson Laboratories) and their substrate. The binding values were corrected by the non-specific binding in the wells containing VWF alone and were expressed as the percentage of residual binding of FVIII. No binding of FVIII was observed on the wells which had not been coated.

### Results: FIG. 5b

Mannan does not interfere in the interaction of FVIII with VWF.

### Example 9

### Investigation of the Internalization of Demannosylated FVIII

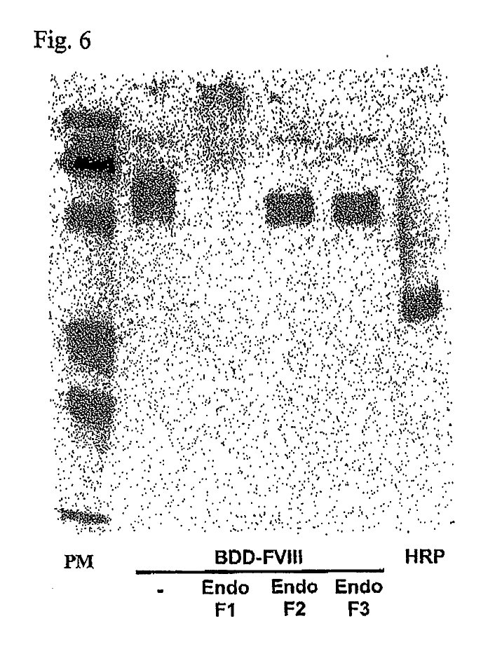

In a first stage, FVIII-BDD is incubated with three different endoglycosidases: EndoF1, EndoF2 and EndoF3 (Sigma). The untreated FVIII-BDD and that treated with EndoF1, EndoF2 and EndoF3 are separated by SDS-PAGE and transferred onto nitrocellulose membrane. The sugars are detected with a glycoprotein detection kit (Sigma) using HRP as a positive control. EndoF1 is the only endoglycosidase which is capable of effectively cutting sugar residues on FVIII-BDD (cf. FIG. 6).

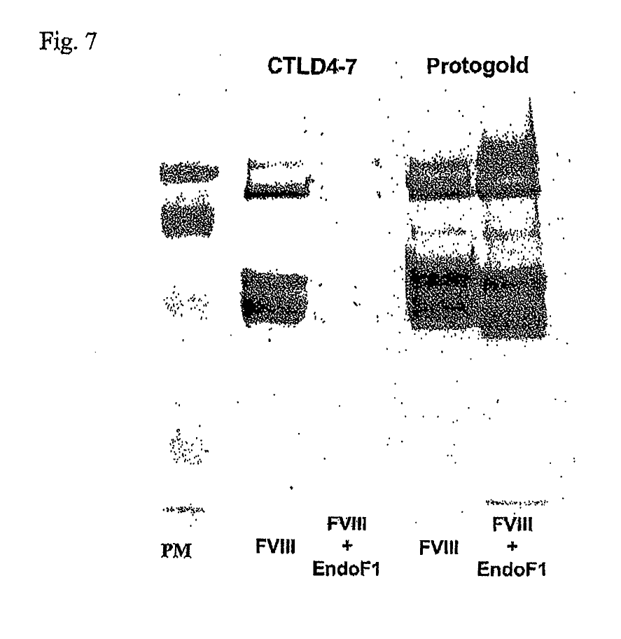

In a second stage, FVIII-BDD is deglycosylated with EndoF1 in accordance with the manufacturer's instructions, separated by SDS-PAGE and revealed by western blotting. 37 μg of FVIII-BDD are loaded into each well of 7.5% SDS-PAGE gels and transferred onto nitrocellulose membranes. These are then revealed using 10 μg/ml of the construct CTLD(4-7)-Fc and an anti-human IgG conjugated to alkaline phosphatase (left) or Protogold (right) (cf. FIG. 7). The modification of the molecular weight of FVIII-BDD after incubation with EndoF1 confirms an effective deglycosylation of EndoF1. Incubation of FVIII-BDD with EndoF1 results in the loss of recognition of FVIII by the construct CTLD(4-7)-Fc, indicating elimination of mannosylated residues.

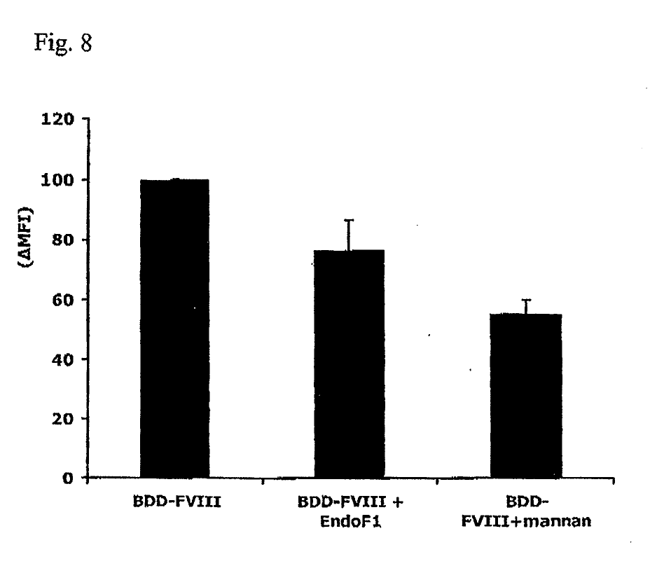

Finally, the inhibition of the internalization of demannosylated FVIII-BDD labelled with FITC was measured. FVIII-BDD conjugated to FITC and treated with EndoF1 (0.143 μM) or untreated is incubated with DCs according to Example 1 (4×10⁵ cells/well of 100 μl) in X-VIVO medium without serum at 37° C. or at 4° C. Beforehand, the cells were preincubated, where appropriate, with mannan (1 mg/ml) for 30 min at 37° C. After incubation for 2 hours, the cells are washed and the mean intensities (mfi) are measured by FACS. The internalization of FVIII-BDD is expressed with respect to the calculated mfi in the presence of untreated FVIII alone (cf. FIG. 8).

The inhibition of the internalization of FVIII-BDD was initially 45±7% with mannan without deglycosylation. It is 23.5±14.4% after incubation with EndoF1. It is reduced further by 32% in the presence of mannan (results not shown), indicating that FVIII treated with EndoF1 is partly internalized in a manner dependent upon the mannose receptor.

This can be explained either:
by the fact that the treatment of FVIII-BDD with EndoF1 only partly removes the mannosylated residues.
or by the fact that EndoF1 leaves N-acetylglucosamine residues, which are also ligands of the mannose receptor.

### Example 10

### Investigation of the Influence of VWF on Recognition of FVIII by the Mannose Receptor

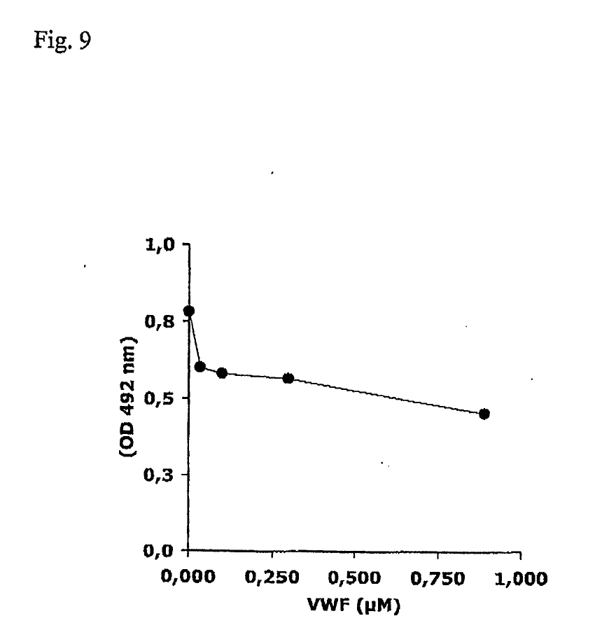

ELISA plates are coated with FVIII-BDD (5.56 μg/ml, 0.033 μM). VWF (0 to 1 μM) is incubated with 10 μg/ml of the construct CTLD(4-7)-Fc for 30 min at room temperature and added to immobilized FVIII-BDD. Binding of the construct to FVIII-BDD is revealed using a mouse anti-human Fc antibody conjugated to HRP and the substrate OPD (cf. FIG. 9).

The addition of soluble VWF prevents CTLD(4-7)-Fc from interacting with the immobilized FVIII to a limited extent (43% inhibition in a VWF:FVIII ratio excess), thus suggesting that the VWF interferes only partly with the recognition of sugar residues on FVIII by the mannose receptor.

### BIBLIOGRAPHY

1. Banchereau, J. and R. M. Steinman, 1998, Dendritic cells and the control of immunity. Nature 392:245.
2. Trombetta, E. S., and I. Mellman, 2005, Cell biology of antigen processing in vitro and in vivo, Annu Rev Immunol 23:975
3. Sallusto, F., M. Cella, C. Danieli and A. Lanzaecchia, 1995, Dendritic cells use macropinocytosis and the mannose receptor to concentrate macromolecules in the major histocompatibility complex class II compartment: downregulation by cytokines and bacterial products, J Exp Med 182:389.
4. Geijtenbeek, T. B., R. Torensma, S. J. van Vliet, G. C. van Duijnhoven, G. J. Adema, Y. van Kooyk and C. G. Figdor, 2000, Identification of DC-SIGN, a novel dendritic cell-specific ICAM-3 receptor that supports primary immune responses, Cell 100:575.
5. Keler, T., V. Ramakrishna and M. W. Fanger, 2004, Mannose receptor-targeted vaccines, Expert Opin Biol Ther 4:1953.
6. Lenting P. J., Neels J. G., van den Berg B. M., Clijsters P. P., Meijerman D. W., Pannekoek H., van Mourik J. A., Mertens K., van Zonneveld A. J., 1999, The light chain of factor VIII comprises a binding site for low density lipoprotein receptor-related protein, J Biol Chem. 274:23734.
7. Sandberg H., Almstedt A., Brandt J., Gray E., Holmquist L., Oswaldsson U., Sebring S., Mikaelsson M., Structural and functional characteristics of the B domain-deleted recombinant factor VIII protein, r-VIII SQ, 2001, Thromb Haemost. 85:93.
8. Kaufman R. J., Wasley L. C., Dorner A. J., Synthesis, processing and secretion of recombinant human factor VIII expressed in mammalian cells, 1988, J Biol Chem 263:6352,
9. Hironaka T., Furukawa K., Esmon P. C., Fournel M. A., Sawada S., Kato M., Minaga T., Kobata A., Comparative study of the sugar chains of factor VIII purified from human plasma and from the culture media of recombinant baby hamster kidney cells. 1992, J Biol Chem. 267:8012,

1.-28. (canceled)

29. Deglycosylated factor VIII or a fragment thereof for which the ability for interacting and the ability for endocytosited by cells which are capable of endocytosing an antigen are decreased or inhibited with respect to native factor VIII.

30. Deglycosylated factor VIII or a fragment thereof according to claim 29, for which the ability for interacting with the receptors present on the surface of the said cells which are capable of endocytosing an antigen is reduced or inhibited.

31. Deglycosylated factor VIII or a fragment thereof according to claim 30, for which the said receptors are receptors specific for mannose.

32. Deglycosylated factor VII or a fragment thereof according to claim 31, for which the said receptors are the mannose receptor CD206 or the DC-SIGN (dendritic cell-specific intercellular adhesion molecule 3 (ICAM-3)-grabbing non-integrin) receptor CD209.

33. Deglycosylated factor VIII or a fragment thereof according to claim 29, for which the said cells which are capable of endocytosing an antigen are antigen-presenting cells (APCs).

34. Deglycosylated factor VIII or a fragment thereof according to claim 33, for which the said antigen-presenting cells (APCs) are dendritic cells or B lymphocytes.

35. Deglycosylated factor VIII or a fragment thereof according to claim 29, for which the said cells which are capable of endocytosing an antigen are chosen from macrophages, endothelial cells, liver sinusoidal endothelial cells, liver Kupffer cells.

36. Deglycosylated factor VIII or a fragment thereof according to claim 29, for which the deglycosylation is obtained without the use of one or more enzymes chosen from the group consisting of a neuraminidase, a beta-galactosidase and an alpha-mannosidase.

37. Deglycosylated factor VIII or a fragment thereof according to claim 29, for which the deglycosylation is obtained by the action of a single enzyme of the endoglucosidase type.

38. Deglycosylated factor VIII or a fragment thereof according to claim 37, for which the said enzyme of the endoglucosidase type is an endo-beta-N-acetylglucosaminidase.

39. Deglycosylated factor VIII or a fragment thereof according to claim 38, for which the said endo-beta-N-acetylglucosaminidase has the ability to cut carbohydrate structures of the oligomannose type and of the hybrid type, and does not have the ability to cut carbohydrate structures of the complex type.

40. Deglycosylated factor VIII or a fragment thereof according to claim 38, for which the said endo-beta-N-acetylglucosaminidase is chosen from the group consisting of endo-beta-N-acetylglucosaminidase F1 and endo-beta-N-acetylglucosaminidase H.

41. Deglycosylated factor VIII or a fragment thereof according to claim 40, for which the said endo-beta-N-acetylglucosaminidase F1 is the endo-beta-N-acetylglucosaminidase F1 of Chryseobacterium (Flavobacterium) meningosepticum.

42. Deglycosylated factor VIII or a fragment thereof according to claim 40, for which the said endo-beta-N-acetylglucosaminidase H is the endo-beta-N-acetylglucosaminidase H of Streptomyces picatus.

43. A pharmaceutical composition comprising a carrier and deglycosylated factor VIII or a fragment thereof according to claim 29.

44. A pharmaceutical composition comprising a carrier and an amount of deglycosylated factor VIII or a fragment thereof according to claim 29 effective to treat a hemophilic patient.

45. A pharmaceutical composition comprising a carrier and an amount of deglycosylated factor VIII or a fragment thereof according to claim 29 effective to treat a patient afflicted with hemophilia of type A or B.

46. A method for treating a hemophilic patient comprising administering to the patient the deglycosylated factor VIII or fragment thereof according to claim 29.

47. The method of claim 46, wherein the factor VIII or fragment is administered in combination with exogenous factor VIII.

48. A method for treating a patient afflicted with hemophilia of type A or B comprising administering to the patient the deglycosylated factor VIII or fragment thereof according to claim 29.

49. A method for treating a hemophilic patient comprising administering to the patient the deglycosylated factor VIII or fragment thereof according to claim 29, wherein the factor VIII or fragment increases the half-life of an exogenous factor VIII in the hemophilic patient.

50. A method for treating a hemophilic patient comprising administering to the patient the deglycosylated factor VIII or fragment thereof according to claim 29, wherein the factor VIII or fragment reduces the immunogenicity of an exogenous factor VIII in the hemophilic patient.

51. Pharmaceutical composition comprising the deglycosylated factor VIII or fragment thereof according to claim 29 and one or more pharmaceutically acceptable adjuvant(s) and/or excipient(s).

52. A method for treating a hemophilic patient comprising administering to the patient a composition comprising mannan.

53. A method for treating a patient afflicted with hemophilia of type A or B comprising administering to the patient a composition comprising mannan.

54. A method for treating a hemophilic patient comprising administering to the patient a composition comprising mannan, wherein the composition increases the half-life of an exogenous factor VIII in a hemophilic patient.

55. A method for treating a hemophilic patient comprising administering to the patient a composition comprising mannan, wherein the composition reduces the immunogenicity of an exogenous factor VIII in a hemophilic patient.

56. The method according to claim 52, wherein the composition comprising mannan also comprises (i) an exogenous factor VIII of the native type and/or (ii) a deglycosylated factor VIII or fragment thereof for which the ability for interacting and the ability for endocytosited by cells which are capable of endocytosing an antigen are decreased or inhibited with respect to native factor VIII

57. The method according to claim 53, wherein the composition comprising mannan also comprises (i) an exogenous factor VIII of the native type and/or (ii) a deglycosylated factor VIII or fragment thereof for which the ability for interacting and the ability for endocytosited by cells which are capable of endocytosing an antigen are decreased or inhibited with respect to native factor VIII

58. The method according to claim 54, wherein the composition comprising mannan also comprises (i) an exogenous factor VIII of the native type and/or (ii) a deglycosylated factor VIII or fragment thereof for which the ability for interacting and the ability for endocytosited by cells which are capable of endocytosing an antigen are decreased or inhibited with respect to native factor VIII

59. The method according to claim 55, wherein the composition comprising mannan also comprises (i) an exogenous factor VIII of the native type and/or (ii) a deglycosylated factor VIII or fragment thereof for which the ability for interacting and the ability for endocytosited by cells which are capable of endocytosing an antigen are decreased or inhibited with respect to native factor VIII
